(19) United States
(12) Patent Application Publication
(10) Pub. No.: US 2021/0169755 A1
(43) Pub. Date: Jun. 10, 2021

(54) DUST REDUCED AMMONIUM FREE BLONDING POWDER

(71) Applicant: Henkel AG & Co. KGaA, Duesseldorf (DE)

(72) Inventors: BERND ANDERHEGGEN, Moenchengladbach (DE); MECHTILD GRUNWALD, Langenfeld (DE)

(73) Assignee: Henkel AG & Co. KGaA, Duesseldorf (DE)

(21) Appl. No.: 17/116,299

(22) Filed: Dec. 9, 2020

(30) Foreign Application Priority Data
Dec. 9, 2019 (DE) 10 2019 219 161.4

(51) Int. Cl. A61K 8/25; A61Q 5/08; A61K 8/02; A61K 8/23; A61K 8/34

(52) U.S. Cl. CPC A61K 8/25; A61Q 5/08; A61K 8/0225; A61K 2800/30; A61K 8/342; A61K 2800/40; A61K 8/23

(57) ABSTRACT

The object of the present disclosure is a blonding powder for mild blonding of keratinous fibers with as natural ingredients as possible, containing at least one oxidizing agent selected from the sodium salts and potassium salts of a peroxosulfuric acid, an oil mixture of at least one branched fatty alcohol with 8-24 carbon atoms and at least one dialkyl ether with 6 to 18 C atoms in the alkyl group, at least one inorganic alkalizing agent which is solid at about 20° C. and 1013 mbar, wherein no ammonium compounds, no paraffin oil, no mineral oil and no silicone compounds are contained.

### CROSS-REFERENCE TO RELATED APPLICATION

This application claims priority to German Patent Application No. 10 2019 219 161.4, filed Dec. 9, 2019, which is incorporated herein by reference in its entirety.

### TECHNICAL FIELD

The present disclosure relates to blonding powders used as brightening agents for keratinous fibers, such as human hair. Furthermore, the present disclosure relates to the use of the agents for gentle bleaching or oxidative brightening of human hair as well as a multi-component packaging unit (kit-of-parts) for brightening keratinous fibers, which comprises a bleaching powder and separately therefrom an oxidizing agent preparation.

Furthermore, a process for the oxidative brightening of keratinous fibers using the said bleaching powder and the kit comprising this bleaching powder is described.

### BACKGROUND

The blonding powder is an anhydrous, powdered oxidizing agent preparation containing at least one persalt.

Lightening one's own hair color has always been the wish of many consumers, as a blonde hair color is considered attractive and desirable in terms of fashion. For this purpose, various bleaching agents with different bleaching power are available on the market. The oxidants contained in these products can lighten the hair fiber by oxidative destruction of the hair's own dye melanin. For a moderate bleaching effect, the use of hydrogen peroxide—possibly with the addition of ammonia or other alkalizing agents—as an oxidizing agent alone is sufficient. A mixture of hydrogen peroxide and at least one compound selected from persalts, especially peroxodisulfate salts and/or peroxomonosulfate salts, is usually used to achieve a stronger bleaching effect. To enhance the blonding effect, the agents contain higher concentrations of hydrogen peroxide and persalts, especially persulfates. Dark, dark brown or black hair can be lightened by from about 4 to about 6 shades in one step. The hydrogen peroxide and the persalts are stored separately until use in order not to deactivate the persalts prematurely. The hydrogen peroxide component, which comprises an aqueous solution of hydrogen peroxide, has an acidic pH, especially a pH of from about 1.5 to about 5.5, especially from about 3 to about 5, measured at about 20° C., to stabilize the hydrogen peroxide.

However, for the melanin-degrading effect of the hydrogen peroxide and the blonding effect on the keratinous fiber, it is advantageous if the application mixture of hydrogen peroxide solution and persalt has an alkaline pH value, preferably in the range of from about 8 to about 12, particularly preferably in the range of from about 8.5 to about 11.5, extremely preferably in the range of from about 9 to about 10.5, measured at about 20° C.

To adjust an alkaline pH value of the brightening application mixture, the blonding powder contains, in addition to at least one persalt, at least one powdery alkalizing agent in such a total amount that the application mixture has the desired alkaline pH value.

If oxidation dye precursors and/or direct dyes are added to the blonding powder, the hair can be dyed at the same time. Appropriate hair dyes are offered especially for consumers with very dark hair rich in melanin.

For safety reasons, the blonding powders should be formulated in such a way that they do not produce, or only produce as little dusting as possible during use, especially before or during mixing with the liquid hydrogen peroxide solution. Suitable features for reducing dust are in particular oils which are mixed with the dust-creating components of the blonding powder, the total amount of oils being from about 0.2-20% by weight, based on the weight of the dusted blonding powder. The application properties of the bleaching powder as well as of the entire ready-to-use bleaching agent can be optimized by skillful selection of the oils. The choice of oils should also take into account the consumer's desire for products that contain as few synthetically produced ingredients as possible, but rather naturally derived ingredients, while also avoiding as far as possible ingredients of mineral origin, such as mineral oils and paraffins.

A further requirement for the blonding powder as contemplated herein was to allow blonding that is as mild as possible Ammonium hydroxide and ammonium salts have therefore been omitted because they can release ammonia in the strongly alkaline medium of the application preparation, which could irritate the scalp and mucous membranes of the user. A further requirement for the blonding powder as contemplated herein was to avoid completely synthetic ingredients as far as possible when selecting the inert carrier components, in particular synthetic ingredients on a petrochemical basis, such as polyacrylates. In contrast, natural ingredients should be preferred, whereby the characteristic “natural” also includes chemical and/or physical modifications of a natural ingredient. Examples are cellulose as a natural polysaccharide and hydroxyethyl cellulose and carboxymethyl celluloses as chemical derivatives of natural cellulose, which are also considered “natural” ingredients for the purposes of this notification.

### BRIEF SUMMARY

Blonding powders and methods of lightening keratinous fibers are provided. In an exemplary embodiment, a blonding powder includes an oxidizing agent that is selected from the group of sodium salts of a peroxosulfuric acid, potassium salts of a peroxosulfuric acid, and mixtures thereof. The blonding powder also includes from about 0.1 to about 15% by weight of a branched fatty alcohol with 8-24 carbon atoms, from about 0.1 to about 15% by weight of a dialkyl ether with 6 to 18 C atoms in the alkyl group, an inorganic alkalizing agent which is solid at about 20° C. and 1013 mbar, and from 0 to about 8% by weight of water, all based on a weight of the blonding powder. A total amount of oils, including the branched fatty alcohol and the dialkyl ether, is from about 0.2 to about 20% by weight, based on the weight of blonding powder. The blonding powder includes no ammonium compounds, no paraffin oil, no mineral oil, and no silicone compounds.

A method of lightening keratinous fibers is provided in another embodiment. The method includes mixing a blonding powder (B) with an oxidizing composition (Ox) to form a ready-to-use mixture, and applying the ready-to-use mixture to the keratinous fibers. The blonding powder (B) includes a) an oxidizing agent selected from the group of sodium salts of a peroxosulfuric acid, potassium salts of a peroxosulfuric acid, and mixtures thereof, b) from about 0.1 to about 15% by weight of a branched fatty alcohol with 8-24 carbon atoms, c) from about 0.1 to about 15% by weight of a dialkyl ether with 6 to 18 C atoms in the alkyl group, d) an inorganic alkalizing agent which is solid at about 20° C. and 1013 mbar, and e) from 0 to about 8% by weight of water, all based on the weight of the blonding powder (B). The blonding powder (B) includes a total amount of oils of from about 0.2 to about 20% by weight, based on the weight of the blonding powder (B), where the total amount of oils includes components b) and c). The blonding powder (B) includes no ammonium compounds, no paraffin oil, no mineral oil, and no silicone compounds. The ready-to-use mixture is left on the keratinous fibers for from about 5 to about 60 minutes, and then the keratinous fibers are rinsed with water and an optional surfactant-containing cleaning agent. The blonding powder (B) and the oxidizing composition (Ox) are in a weight ratio (B) : (Ox) of from about 0.2-1.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

Surprisingly, these tasks were solved by the object of the claims. As contemplated herein, keratinous fibers are defined as furs, wool, feathers, and especially human hair. Although the agents as contemplated herein are primarily suitable for bleaching and/or lightening fibers containing keratin, there is nothing in principle to prevent their use in other areas.

A first object of the present disclosure is a blonding powder comprising
a) at least one oxidizing agent selected from the sodium salts and potassium salts of a peroxosulfuric acid, and mixtures thereof,
b) furthermore from about 0.1-15% by weight of at least one branched fatty alcohol with 8-24 carbon atoms, each based on the weight of the blonding powder,
c) furthermore from about 0.1-15% by weight of at least one dialkyl ether with 6 to 18 C atoms in the alkyl group, in each case based on the weight of the blonding powder,
d) furthermore at least one inorganic alkalizing agent which is solid at about 20° C. and 1013 mbar,
e) and from 0 to about 8% by weight, preferably from about 0.1 to about 5% by weight, particularly preferably from about 0.5 to about 3% by weight of water, in each case based on the weight of the blonding powder,
f) wherein the total amount of oils, including components b) and c), is from about 0.2-20% by weight, based on the weight of the blonding powder, and wherein the blonding powder includes no ammonium compounds, no paraffin oil, no mineral oil, and no silicone compounds.

As contemplated herein, the term “powder” or “powdery” means a solid, free-flowing dosage form including individual particles, solid at about 20° C. and 1013 mbar, in which the individual particles have particle sizes ranging from about 0.1 μm to a maximum of about 1.6 mm The particle size distribution can preferably be determined by laser diffraction measurement according to ISO 13320-1 (2009). If necessary, the particle size of the blonding powder can be adjusted by physical treatment, such as sieving, pressing, granulating or pelletizing, or by the addition of certain auxiliary substances, to meet the requirements of the blonding powder, e.g. to improve the miscibility of the individual powder components or the miscibility of the blonding powder with a hydrogen peroxide preparation.

Blonding powders preferred according to the present disclosure have a bulk density in the range of from about 400 to about 1000 g/l (grams/liter), preferably from about 450 to about 900 g/l, especially preferred from about 550 to about 820 g/l. The bulk density is preferably determined according to EN ISO 60 (version 01/2000) or DIN ISO 697 (version 01/1984).

Unless otherwise stated, all temperature specifications refer to a pressure of 1013 mbar.

The term “ammonium salt” refers to salts with the cation NH4+ and an anionic counter ion. Quaternary ammonium compounds in which one or more H atoms on the nitrogen atom are replaced by an organic group, e.g. an alkyl group, in particular tetraalkylammonium-based surfactants, are not covered by the term “ammonium salt”.

The blonding powder as contemplated herein contains as the first essential component at least one oxidizing agent selected from inorganic salts of a peroxosulfuric acid and mixtures thereof.

Peroxosulfuric acids are peroxodisulfuric acid and peroxomonosulfuric acid (Caro's acid).

Preferably at least one inorganic salt of peroxosulfuric acid is selected from alkali metal peroxodisulfates, alkali metal peroxomonosulfates and alkali metal hydrogen peroxomonosulfates. Potassium peroxodisulfate, sodium peroxodisulfate and potassium hydrogen peroxomonosulfate are particularly preferred. Furthermore, during the work on the present disclosure, it has proved to be particularly preferred if the blonding powder as contemplated herein contains at least two different peroxodisulfates. Preferred peroxodisulfate salts are mixtures of potassium peroxodisulfate and sodium peroxodisulfate.

Preferred blonding powders as contemplated herein contain at least one oxidizing agent selected from inorganic salts of a peroxosulfuric acid and mixtures thereof in a total amount of from about 5-85% by weight, preferably from about 10-70% by weight, particularly preferably from about 17-55% by weight, extremely preferably from about 22-45% by weight, in each case based on the weight of the blonding powder.

The blonding powder as contemplated herein contains as a second essential constituent from about 0.1-15% by weight of at least one branched fatty alcohol with 8-24 carbon atoms, in each case based on the weight of the blonding powder, as oil component b). The branched alcohols are often referred to as Guerbet alcohols because they are obtained by the Guerbet reaction. Preferred branched fatty alcohols with 8-24 carbon atoms are selected from 2-octyldodecanol, 2-hexyldecanol, 2-ethylhexyl alcohol, isostearyl alcohol, and mixtures thereof. As contemplated herein, 2-octyldodecanol is extremely preferred as oil component b). The at least one branched fatty alcohol with 8-24 carbon atoms is contained in a total amount of from about 0.1-15% by weight, preferably from about 1-13% by weight, particularly preferably from about 5-12% by weight, each based on the weight of the blonding powder. Extraordinarily preferred blonding powders are exemplified by a content of from about 0.1-15% by weight, preferably from about 1-13% by weight, particularly preferably from about 5-12% by weight, of 2-octyldodecanol, in each case based on the weight of the blonding powder.

The blonding powder as contemplated herein contains as a third essential constituent from about 0.1-15% by weight of at least one dialkyl ether with 6 to 18 C atoms in the alkyl group, based on the weight of the blonding powder, as oil component c).

As oil component c), particularly preferred dialkyl ethers are di-n-alkyl ethers with a total of 12 to 36 C atoms, especially 16 to 24 C atoms in the molecule, such as di-n-octyl ether, di-n-decyl ether, di-n-nonylether, di-n-undecyl ether, di-n-dodecyl ether, di-n-octadecyl ether, n-hexyl-n-octyl ether, n-octyl-n-decyl ether, n-decyl-n-undecyl ether, n-undecyl-n-dodecyl ether and n-hexyl-n-undecyl ether. Also preferred dialkyl ethers are those with branched alkyl groups each with 6 to 18 C atoms, especially with alkyl groups substituted in the 2-position with an ethyl group. Preferred branched-chain dialkyl ethers with 6 to 18 C atoms in the alkyl group are selected from di-(2-ethylhexyl)ether and di-(2-ethyldecyl)ether.

Di-n-octylether is highly preferred (INCI: Dicaprylyl ether), which is commercially available for example under the name Cetiol® OE.

The at least one dialkyl ether with 6 to 18 C atoms in the alkyl group is contained in a total amount of from about 0.1-15% by weight, preferably from about 0.5-10% by weight, particularly preferably from about 1-5% by weight, extremely preferably from about 2-3% by weight, each based on the weight of the blonding powder. Extraordinarily preferred blonding powders are exemplified by a content of from about 0.1-15% by weight, preferably from about 0.5-10% by weight, particularly preferably from about 1-5% by weight, extremely preferably from about 2-3% by weight of di-n-octyl ether, in each case based on the weight of the blonding powder.

In blonding powders which are particularly preferred as contemplated herein, the weight ratio of the total amount of branched fatty alcohols with 8-24 carbon atoms to the total amount of dialkyl ethers with 6 to 18 carbon atoms in the alkyl group is 2-8, preferably 3-6. In blonding powders, which as contemplated herein are extremely preferred, the weight ratio of 2-octyldodecanol to di-n-octylether is 2-8, preferably 3-6.

The total amount of oils, including components b) and c), is from about 0.2-20% by weight, preferably from about 1-17% by weight, particularly preferably from about 3-16% by weight, extremely preferably from about 5-15% by weight, further extremely preferably from about 9-14% by weight, each based on the weight of the blonding powder.

The blonding powder as contemplated herein contains as a fourth essential component at least one inorganic alkalizing agent which is solid at about 20° C. and 1013 mbar.

Blonding powders preferred as contemplated herein contain at least one inorganic alkalizing agent that is solid at about 20° C. and 1013 mbar, preferably in a total amount of from about 4-70% by weight, preferably from about 10-65% by weight, particularly preferably from about 15-60% by weight, extremely preferably from about 20-55% by weight, in each case based on the weight of the blonding powder.

Blonding powders preferred as contemplated herein additionally contain at least one inorganic alkalizing agent which is solid at about 20° C. and 1013 mbar, in a total amount of from about 0.1 to about 50% by weight, preferably from about 4 to about 30% by weight, particularly preferably from about 15-25% by weight, in each case based on the weight of the blonding powder, where the alkalizing agent includes at least one sodium silicate or sodium metasilicate with a molar SiO₂/Na₂O ratio of ≥2, preferably from about 2.5-3.5.

In addition to the at least one sodium silicate or sodium metasilicate with a molar SiO₂/Na₂O ratio of ≥2, preferably from about 2.5-3.5, in a total amount of from about 0.1-50 wt. %, preferably from about 4-30 wt. %, particularly preferably from about 15-25 wt.-%, in each case based on the weight of the blonding powder, optional alkalizing agents further include inorganic alkalizing agents selected from alkaline earth metal silicates, which are solid at about 20° C. and 1013 mbar and are particularly preferred. As contemplated herein, the inorganic alkalizing agents may include alkaline earth metal hydroxide carbonates, alkaline earth metal carbonates, alkaline earth metal metasilicates, alkali metal hydroxides, alkaline earth metal hydroxides, (earth) alkali metal phosphates, (earth) alkali metal hydrogen phosphates, and mixtures of these substances. As contemplated herein, particularly preferred inorganic alkalizing agents which are solid at about 20° C. and 1013 mbar are, in addition to the at least one obligatory sodium silicate or sodium metasilicate, each with a molar SiO_{2/}Na₂O ratio of ≥2, preferably from about 2.5 to about 3.5, selected from magnesium hydroxide carbonates and mixtures of these alkalizing agents. As contemplated herein, preferred magnesium hydroxide carbonates are those with the formula MgCO₃.Mg(OH)₂.2 H₂O and those with the formula MgCO₃.g(OH)₂. Magnesium hydroxide carbonate with the formula MgCO₃.Mg(OH)₂ is particularly preferred as contemplated herein.

Blonding powders particularly preferred as contemplated herein contain, in each case based on their total weight, from about 0.1 to 50% by weight, preferably from about 4-30% by weight, particularly preferably from about 15-25% by weight, of sodium silicates with a molar SiO₂/Na₂O ratio of ≥2, preferably from about 2.5 to about 3.5, and from about 2-40% by weight, preferably from about 5-35% by weight, particularly preferably from about 10-32% by weight, of magnesium hydroxide carbonate as inorganic alkalizing agents solid at about 20° C. and 1013 mbar.

Blonding powders of extraordinary preference as contemplated herein contain, in each case based on their total weight, from about 0.1 to about 50 wt. %, preferably from about 4-30 wt. %, particularly preferably from about 15-25 wt. %, sodium silicates with a molar SiO₂/Na₂O ratio of ≥2, preferably from about 2.5 to about 3.5, and from about 2-40 wt. %, preferably from about 5-35% by weight, particularly preferably from about 10-32% by weight, of magnesium hydroxide carbonate having the formula MgCO₃.Mg(OH)₂ as inorganic alkalizing agents solid at about 20° C. and 1013 mbar.

The blonding powders as contemplated herein are anhydrous, which in the sense of the present disclosure means that they contain from 0 to about 8% by weight, preferably from about 0.1 to about 5% by weight, particularly preferably from about 0.5 to about 3% by weight, of water, each based on the weight of the blonding powder.

These figures refer to the content of free water. Not considered is the content of molecularly bound water or water of crystallization, which individual powder components may have. The water content can be determined by Karl Fischer titration according to ISO 4317 (Version 2011-12).

The blonding powders as contemplated herein are further exemplified by the fact that they contain no ammonium compounds, no paraffin oil, no mineral oil, and no silicone compounds.

In addition to the mandatory oil components b) and c), the blonding powder as contemplated herein may contain at least one additional oil, provided that the total amount of oils, including components b) and c), is in the range of from about 0.2-20% by weight, based on the weight of the blonding powder, and the additional oil is not a paraffin oil, mineral oil or silicone compound.

Preferably, the total amount of optional oils is from about 0.05-5% by weight, particularly preferably from about 0.1-3% by weight, extremely preferably from about 0.2-1% by weight, each based on the weight of the blonding powder.

As contemplated herein, preferred optional oils are selected from the benzoic acid esters of linear or branched C8-22 alkanols. Benzoic acid C12-C15 alkyl esters are particularly preferred.

Other optional oils preferred by the present disclosure are selected from the triglycerides (=triple esters of glycerol) of linear or branched, saturated or unsaturated, optionally hydroxylated C8-30 fatty acids. Natural oils that may be selected from, e.g. amaranth seed oil, apricot kernel oil, argan oil, avocado oil, babassu oil, cotton seed oil, borage seed oil, camelina oil, thistle oil, peanut oil, pomegranate kernel oil, grapefruit seed oil, hemp oil, hazelnut oil, elderberry seed oil, currant seed oil, jojoba oil, linseed oil, macadamia nut oil, corn germ oil, almond oil, marula oil, evening primrose oil, olive oil, palm oil, palm kernel oil, Brazil nut oil, pecan oil, peach kernel oil, rapeseed oil, castor oil, sea buckthorn pulp oil, sea buckthorn seed oil, sesame oil, soybean oil, sunflower oil, grape seed oil, walnut oil, wild rose oil, wheat germ oil, and the liquid portions of coconut oil and the like. Preferred are saturated triglyceride oils, especially Capric/Caprylic Triglycerides.

Other optional oils which are particularly preferred as contemplated herein are selected from the dicarboxylic acid esters of linear or branched C2-C10 alkanols, in particular diisopropyl adipate, di-n-butyl adipate, di-(2-ethylhexyl) adipate, dioctyl adipate, diethyl/di-n-butyl/dioctyl sebacate, diisopropyl sebacate, dioctyl malate, dioctyl maleate, dicaprylyl maleate, diisooctyl succinate, di-2-ethylhexyl succinate and di-(2-hexyldecyl) succinate.

Other optional oils which are particularly preferred by the present disclosure are selected from the esters of linear or branched saturated or unsaturated fatty alcohols with 2-30 carbon atoms, and with linear or branched saturated or unsaturated fatty acids with 2-30 carbon atoms, which may be hydroxylated. These are preferably 2-hexyl decyl stearate, 2-hexyl decyl laurate, isodecyl neopentanoate, isononyl isononanoate, 2-ethylhexyl palmitate and 2-ethylhexyl stearate, isopropyl myristate, isopropyl palmitate, isopropyl stearate, isopropyl isostearate, isopropyl oleate, isooctyl stearate, isononyl stearate, isocetylstearate, isononylisononanoate, isotridecylisononanoate, cetearylisononanoate, 2-ethylhexyl laurate, 2-ethylhexyl isostearate, 2-ethylhexyl cocoate, 2-octyldodecyl palmitate, butyloctanoic acid 2-butyloctanoate, diisotridecyl acetate, n-butyl stearate, n-hexyl laurate, n-decyl oleate, oleyl oleate, oleylerucate, erucyl oleate, erucylerucate, ethylene glycol dioleate and ethylene glycol dipalmitate.

Other optional oils preferred by the present disclosure are selected from the C₈-C₂₂ fatty alcohol esters of monovalent or polyvalent C₂-C₇ hydroxycarboxylic acids, especially the esters of glycolic acid, lactic acid, malic acid, tartaric acid, citric acid and salicylic acid, e.g. C₁₂-C₁₅ alkyl lactate.

Other optional oils preferred by the present disclosure are selected from the symmetrical, asymmetrical or cyclic esters of carbonic acid with C₃₋₂₂ alkanols, C₃₋₂₂ alkane diols or C₃₋₂₂ alkane triols, e.g. dicaprylylcarbonate, or the esters according to DE19756454 A1, especially glycerine carbonate.

Preferred blonding powders as contemplated herein may include an optional oil that is selected from the benzoic acid esters of linear or branched C₈₋₂₂ alkanols; triglycerides of linear or branched, saturated or unsaturated, optionally hydroxylated C₈₋₃₀ fatty acids, especially natural oils; the dicarboxylic acid esters of linear or branched C₂-C₁₀ alkanols; the esters of linear or branched saturated or unsaturated fatty alcohols containing 2-30 carbon atoms with linear or branched saturated or unsaturated fatty acids containing 2-30 carbon atoms, which may be hydroxylated; the C₈-C₂₂ fatty alcohol esters of monovalent or polyvalent C₂-C₇ hydroxycarboxylic acids; the symmetrical, asymmetrical or cyclic esters of carbonic acid with C₃₋₂₂ alkanols, C₃₋₂₂ alkanediols or C₃₋₂₂ alkanediols; the esters of dimers of unsaturated C₁₂-C₂₂ fatty acids (dimer fatty acids) with monovalent linear, branched or cyclic C₂-C₁₈ alkanols or with polyvalent linear or branched C₂-C₆ alkanols, and mixtures of the above substances.

A blonding powder preferred as contemplated herein still contains hydroxyethyl cellulose (INCI) quaternized with diallyldimethylammonium chloride as an optional component. Polyquaternium-4 is particularly preferably contained in a total amount of from about 0.010-0.500 wt. %, particularly preferably from about 0.050-0.300 wt. %, further particularly preferably from about 0.090-0.200, extremely preferably from about 0.095-0.150 wt. %, each based on the weight of the blonding powder.

A blonding powder preferred as contemplated herein also contains 2-[2-hydroxy-3-(trimethylammonio)propoxy]ethyl cellulose ether chloride (INCI) as an optional component. The 2-[2-hydroxy-3-(trimethylammonio)propoxy]ethyl cellulose ether chloride is particularly preferably contained in a total amount of from about 0.01-1.00% by weight, particularly preferably from about 0.10-0.60% by weight, further particularly preferably from about 0.20-0.48, extremely preferably from about 0.30-0.47% by weight, in each case based on the weight of the blonding powder.

A further blonding powder preferred as contemplated herein further contains as an optional component at least one amino acid selected from arginine, lysine, histidine or at least one of the salts of these amino acids. Arginine is highly preferred. As contemplated herein, mixtures of arginine and lysine may be particularly preferred. Among the salts of arginine, lysine or histidine preferred by the present disclosure are the alkali metal salts and alkaline earth metal salts, in particular the salts of lithium, sodium, potassium, magnesium and calcium, as well as hydrohalides, in particular hydrochlorides, and mixtures of these salts. As contemplated herein, lysine hydrochloride is a particularly preferred amino acid salt.

As contemplated herein, particularly preferred amino acid mixtures are selected from arginine/lysine hydrochloride and arginine/lysine.

The amino acids, selected from arginine, lysine, histidine, and their salts as contemplated herein, may also contain water of crystallization.

Blonding powders of extraordinary preference as contemplated herein contain at least one amino acid selected from arginine, lysine, histidine or at least one salt of these amino acids in a total amount, converted to the mass of free amino acid, of from about 0.1-7% by weight, preferably from about 0.2-5% by weight, particularly preferably from about 0.5-2.5% by weight, extremely preferably from about 1-2% by weight, in each case based on the weight of the blonding powder.

In a preferred form, the blonding powder as contemplated herein further contains at least one dicarboxylic acid with 2 to 10 carbon atoms, which is particularly preferably selected from succinic acid, malic acid, oxalic acid, malonic acid, adipic acid, pimelic acid, cork acid, azelaic acid, sebacic acid, maleic acid, fumaric acid, D-tartaric acid, L-tartaric acid, meso-tartaric acid, racemic acid, alpha-ketoglutaric acid, beta-ketoglutaric acid, oxaloacetic acid, and/or at least one salt of these acids, as well as mixtures of these compounds, wherein the at least one dicarboxylic acid with 2 to 10 carbon atoms being selected extremely preferably from succinic acid, malic acid and maleic acid, as well as their salts.

Salts of dicarboxylic acids with 2 to 10 carbon atoms preferred by the present disclosure are selected from the mono- and disalts of the anions of succinic acid, malic acid, oxalic acid, malonic acid, adipic acid, pimelic acid, cork acid, azelaic acid, sebacic acid, maleic acid, fumaric acid, D-tartaric acid, L-tartaric acid, meso-tartaric acid, racemic acid, alpha-ketoglutaric acid, beta-ketoglutaric acid and oxaloacetic acid with alkali metal ions, alkaline earth metal ions and the ions of basic amino acids such as arginine, lysine and histidine, in particular with lithium, sodium, potassium, magnesium and calcium ions, and mixtures of these salts.

Succinic acid, which as contemplated herein is particularly preferred, has a melting point in the range of from about 185-187° C. at 1013 mbar and is therefore a solid at about 20° C. Succinic acid salts suitable as contemplated herein are selected from the succinates and hydrogen succinates of alkali metal ions, alkaline earth metal ions and the ions of basic amino acids, such as arginine, lysine and histidine, in particular the lithium, sodium, potassium, magnesium and calcium ions, or the succinates and hydrogen succinates of basic amino acids, such as arginine, lysine and/or histidine, e.g. arginine succinate, and mixtures of these salts. The salts of succinic acid may also contain bound water of crystallization. Sodium succinate hexahydrate, as contemplated herein, is particularly preferred.

As contemplated herein, malic acid, which is particularly preferred, is optically active. The racemic DL-malic acid has a melting point at 1013 mbar in the range of from about 131-132° C., so at about 20° C. it is a solid. The enantiomers D-malic acid and L-malic acid each have a melting point in the range of from about 100-101° C. at 1013 mbar. For cost reasons, racemic DL-malic acid is preferred.

Salts of malic acid suitable as contemplated herein are selected from the malates and hydrogen malates of alkali metal ions, alkaline earth metal ions and the ions of basic amino acids, such as arginine, lysine and histidine, especially lithium, sodium, potassium, magnesium and calcium ions, as well as mixtures of these salts, especially disodium malate and dipotassium malate, but also calcium malate. The abovementioned salts of malic acid, suitable as contemplated herein, may contain bound water of crystallization, in particular disodium malate hemihydrate and disodium malate trihydrate.

Oxalic acid, which is preferred as contemplated herein, has a melting point of about 189.5° C. (anhydrous) at 1013 mbar or, as a dihydrate, a melting point of about 101.5° C. Oxalic acid salts suitable as contemplated herein are selected from the oxalates and hydrogen oxalates of alkali metal ions, alkaline earth metal ions and the ions of basic amino acids, such as arginine, lysine and histidine, in particular lithium, sodium, potassium, magnesium and calcium ions, as well as mixtures of these salts.

Malonic acid, which is the preferred in the present disclosure, has a melting point of about 135° C. at 1013 mbar. Salts of malonic acid which are suitable as contemplated herein are selected from the malates and hydrogen malates of alkali metal ions, alkaline earth metal ions and the ions of basic amino acids, such as arginine, lysine and histidine, in particular of lithium, sodium, potassium, magnesium and calcium ions, as well as mixtures of these salts.

The adipic acid preferred in the present disclosure has a melting point of about 152° C. at 1013 mbar. Salts of adipic acid which are suitable as contemplated herein are selected from the adipates and hydrogen adipates of alkali metal ions, alkaline earth metal ions and the ions of basic amino acids, such as arginine, lysine and histidine, especially lithium, sodium, potassium, magnesium and calcium ions, as well as mixtures of these salts.

Pimelic acid, which is preferred as contemplated herein, has a melting point of about 105° C. at 1013 mbar. Salts of pimelic acid suitable as contemplated herein are selected from the pimelates and hydrogen pimelates of alkali metal ions, alkaline earth metal ions and the ions of basic amino acids, such as arginine, lysine and histidine, in particular lithium, sodium, potassium, magnesium and calcium ions, as well as mixtures of these salts.

As contemplated herein, the preferred cork acid has a melting point of 144° C. at 1013 mbar. Salts of cork acid suitable as contemplated herein are selected from the suberates and hydrogen suberates of alkali metal ions, alkaline earth metal ions and the ions of basic amino acids, such as arginine, lysine and histidine, especially lithium, sodium, potassium, magnesium and calcium ions, and mixtures of these salts.

Azelaic acid, which is the preferred acid as contemplated herein, has a melting point of about 106° C. at 1013 mbar. Salts of azelaic acid which are suitable as contemplated herein are selected from the azelates and hydrogen azelates of alkali metal ions, alkaline earth metal ions and the ions of basic amino acids, such as arginine, lysine and histidine, especially lithium, sodium, potassium, magnesium and calcium ions, as well as mixtures of these salts.

Sebacic acid, which is preferred as contemplated herein, has a melting point of about 134.5° C. at 1013 mbar. Salts of sebacic acid which are suitable as contemplated herein are selected from the sebacates and hydrogen sebacates of alkali metal ions, alkaline earth metal ions and the ions of basic amino acids, such as arginine, lysine and histidine, especially lithium, sodium, potassium, magnesium and calcium ions, as well as mixtures of these salts.

Maleic acid, which as contemplated herein is particularly preferred, has a melting point of from about 130 to about 131° C. at 1013 mbar (from ethanol or benzene) and of from about 138 to about 139° C. (from water). Salts of maleic acid suitable as contemplated herein are selected from the maleates and hydrogen maleates of alkali metal ions and alkaline earth metal ions, of lithium, sodium, potassium, magnesium and calcium ions, and mixtures of these salts.

Fumaric acid, which as contemplated herein is particularly preferred, has a melting point of about 287° C. at 1013 mbar in the sealed tube: at about 200° C., fumaric acid sublimates. Salts of fumaric acid suitable as contemplated herein are selected from the fumarates and hydrogen fumarates of alkali metal ions and alkaline earth metal ions, of lithium, sodium, potassium, magnesium and calcium ions, and mixtures of these salts.

The D-tartaric acid (anticlockwise), which is particularly preferred as contemplated herein, has a melting point of from about 168-170° C. at 1013 mbar. Salts of D-tartaric acid suitable as contemplated herein are selected from the tartrates and hydrogentartrates of alkali metal ions and alkaline earth metal ions, especially lithium, sodium, potassium, magnesium and calcium ions, as well as mixtures of these salts.

The L-tartaric acid (clockwise rotation), which is particularly preferred as contemplated herein, has a melting point of from about 168-170° C. at 1013 mbar. Salts of L-tartaric acid suitable as contemplated herein are selected from the tartrates and hydrogentartrates of alkali metal ions and alkaline earth metal ions, especially lithium, sodium, potassium, magnesium and calcium ions, as well as mixtures of these salts.

The meso-tartaric acid, which as contemplated herein is particularly preferred, has a melting point of about 140° C. at 1013 mbar. Salts of meso-tartaric acid suitable as contemplated herein are selected from the tartrates and hydrogentartrates of alkali metal ions and alkaline earth metal ions, in particular lithium, sodium, potassium, magnesium and calcium ions, as well as mixtures of these salts.

As contemplated herein, the racemic mixture of D-tartaric acid and L-tartaric acid is the most preferred grape acid. Grape acid has a melting point of about 206° C. at 1013 mbar. Salts of grape acid suitable as contemplated herein are selected from the tartrates and hydrogentartrates of alkali metal ions and alkaline earth metal ions, of lithium, sodium, potassium, magnesium, and calcium ions, as well as mixtures of these salts.

The alpha-ketoglutaric acid, which as contemplated herein is particularly preferred, has a melting point of from about 112-116° C. at 1013 mbar. Salts of alpha-ketoglutaric acid suitable as contemplated herein are selected from the alpha-ketoglutarates and alpha-ketohydrogen glutarates of alkali metal ions and alkaline earth metal ions, in particular of lithium, sodium, potassium, magnesium and calcium ions, as well as mixtures of these salts.

Beta-ketoglutaric acid, which as contemplated herein is particularly preferred, has a melting point of about 122° C. at 1013 mbar; it melts with decomposition. Salts of beta-ketoglutaric acid suitable as contemplated herein are selected from the beta-ketoglutarates and beta-ketohydrogen glutarates of alkali metal ions and alkaline earth metal ions, in particular of lithium, sodium, potassium, magnesium and calcium ions, and mixtures of these salts.

Oxaloacetic acid, which is particularly favored by the present disclosure, has a melting point of about 161° C. at 1013 mbar. Salts of oxaloacetic acid which are suitable as contemplated herein are selected from the oxaline acetates and oxaline hydrogen acetates of alkali metal ions and alkaline earth metal ions, in particular of lithium, sodium, potassium, magnesium and calcium ions, as well as mixtures of these salts.

Blonding powders preferred as contemplated herein, contain at least one dicarboxylic acid with 2 to 10 carbon atoms, selected from succinic acid, malic acid, oxalic acid, malonic acid, adipic acid, pimelic acid, cork acid, azelaic acid, sebacic acid, and/or at least one salt of these acids, in a total amount of from about 0.03-7 wt. %, converted to the mass of free dicarboxylic acid. %, preferably from about 0.1-5 wt. %, particularly preferably from about 0.5-3 wt. %, extremely preferably from about 0.9-1.5 wt. %, each based on the weight of the blonding powder.

Further blonding powders preferred as contemplated herein contain succinic acid and/or at least one salt of succinic acid in a total amount, converted to the mass of free dicarboxylic acid, of from about 0.03-7% by weight, preferably from about 0.1-5% by weight, particularly preferably from about 0.5-3% by weight, extremely preferably from about 0.9-1.5% by weight, in each case based on the weight of the blonding powder.

Further blonding powders preferred as contemplated herein contain malic acid and/or at least one salt of malic acid in a total amount, converted to the mass of free dicarboxylic acid, of from about 0.03-7% by weight, preferably from about 0.1-5% by weight, particularly preferably from about 0.5-3% by weight, extremely preferably from about 0.9-1.5% by weight, in each case based on the weight of the blonding powder.

Blonding powders particularly preferred as contemplated herein further contain at least one or more hydrophilic thickeners, preferably selected from polysaccharides, which may be chemically and/or physically modified. As contemplated herein, compounds from the group of polysaccharides are particularly preferred as hydrophilic thickeners, as the basic structures of the polysaccharides are of natural origin and biodegradable. Preferred hydrophilic polysaccharide thickeners are selected from celluloses, cellulose ethers of C1-C4 alcohols, cellulose esters, xanthan gum, alginic acids (and their corresponding physiologically compatible salts, the alginates), agar agar (with the polysaccharide agarose as the main component present in agar agar), starch fractions and starch derivatives such as amylose, amylopectin and dextrins, karaya gum, locust bean gum, gum arabic, pectins, dextran and guar gum and mixtures thereof.

Cellulose ethers of C1-C4 alcohols and cellulose esters preferred as contemplated herein are selected from methyl celluloses, ethyl celluloses, hydroxyalkyl celluloses (such as hydroxyethyl cellulose), methyl hydroxyalkyl celluloses and carboxymethyl celluloses (such as those with the INCI designation cellulose gum) and their physiologically compatible salts.

Carboxymethyl cellulose (preferably carboxymethyl cellulose with the INCI designation cellulose gum) is contained in preferred forms as a hydrophilic thickener for reliable viscosity adjustment and residue-free application on keratin fibers and the scalp. Carboxymethyl cellulose may be present in a preferred form as the sole hydrophilic thickener. A combination of carboxymethylcellulose and hydroxyethylcellulose is particularly preferred.

A combination of carboxymethyl cellulose and xanthan (preferably xanthan with the INCI designation xanthan gum) may also be preferred as contemplated herein.

Blonding powders particularly preferred as contemplated herein contain at least one hydrophilic thickener in a total amount of from about 0.1 to about 5% by weight, preferably from about 0.5 to about 4% by weight, more preferably from about 1 to about 3.5% by weight and very particularly preferably from about 2.5 to about 3.4% by weight, in each case based on the weight of the blonding powder.

In a further preferred embodiment of the present disclosure, the blonding powder as contemplated herein contains, in each case based on its weight, from about 0.1 to about 4% by weight, preferably from about 1 to about 2.8% by weight, of carboxymethyl cellulose.

In a further preferred embodiment of the present disclosure, the blonding powder as contemplated herein contains, in each case based on its weight, from about 0.1 to about 3% by weight, preferably from about 0.5 to about 2.5% by weight, more preferably from about 0.7 to about 1.5% by weight, of hydroxyethyl cellulose.

In a further preferred embodiment of the present disclosure, the blonding powder as contemplated herein contains, in each case based on its weight, from about 0.1 to about 3% by weight, preferably from about 0.5 to about 2.5% by weight, more preferably from about 0.7 to about 1.5% by weight, of xanthan gum.

A further object of the present disclosure is a process for brightening keratinous fibers, in particular human hair, in which a blonding powder as contemplated herein is mixed with an oxidation composition to form a ready-to-use mixture, where the oxidation composition , in each case based on its weight, contains from about 50-96% by weight, preferably from about 70-93% by weight, particularly preferably from about 80-90% by weight, water and from about 0.5-20% by weight of a water-based oxidation agent. The water-based oxidation agent may be hydrogen peroxide, and the oxidation composition may further contain at least one pH adjusting agent in such an amount that the oxidation composition has a pH value in the range of from about 1.5 to about 5.5, measured at about 20° C. The ready-to-use mixture is applied to the keratin-containing fibers immediately after preparation, is left on the fibers for from about 5 to about 60 minutes, and then the fibers are rinsed with water and optionally washed out with a surfactant-containing cleaning agent. The blonding powder (B) and the oxidation composition (Ox) preferably are present in a ratio (B) : (Ox) of from about 0.2-1, particularly preferred from about 0.3-0.8, further preferred from about 0.4-0.7, and extremely preferred from about 0.5-0.6.

The oxidation composition (Ox) used in the whitening process as contemplated herein essentially contains water and hydrogen peroxide. The concentration of hydrogen peroxide is determined on the one hand by the legal requirements and on the other hand by the desired effect. It is from about 0.5-20% by weight, preferably from about 3-12% by weight, particularly preferably from about 6-9% by weight of hydrogen peroxide (calculated as 100% H2O2), each based on the weight of the oxidation composition (Ox).

To stabilize the hydrogen peroxide, the oxidation composition (Ox) preferably has an acidic pH value, in particular a pH value in the range of from about 1.5 to about 5.5, measured at about 20° C. To stabilize the hydrogen peroxide, it also preferably contains complexing agents, preservatives, stabilizing agents and/or buffer substances.

As contemplated herein, the blonding powder is preferably composed in such a way that the mixture with the aforementioned oxidation composition (Ox), i.e. the brightening agent ready-to-use mixture, has an alkaline pH value, preferably a pH value of from about 8.5 to about 12, particularly preferably a pH value of from about 9.0 to about 11.5, extremely preferably a pH value of from about 9.5 to about 11.0, in each case measured at about 20° C.

Oxidation compositions (Ox) used with particular preference as contemplated herein further contain at least one oil and/or at least one fat component with a melting point in the range of from about 23-110° C., preferably in a total amount of from about 0.1-60% by weight, particularly preferably from about 0.5-40% by weight, extremely preferably from about 2-24% by weight, in each case based on the weight of the oxidation composition (Ox) preferably used as contemplated herein.

The oils suitable for the oxidation compositions (Ox) preferred by the present disclosure are, for the most part, the same oils that are revealed above as suitable dust removal agents.

Oxidizing compositions (Ox) particularly preferred as contemplated herein contain at least one oil selected from the benzoic acid esters of linear or branched C8-22 alkanols. Benzoic acid C12-C15 alkyl esters are particularly preferred.

Other oils suitable for oxidation compositions (Ox), which as contemplated herein are particularly preferred, are selected from the triglycerides (=triple esters of glycerol) of linear or branched, saturated, optionally hydroxylated C8-30 fatty acids. Preferred are saturated triglyceride oils, especially Capric/Caprylic Triglycerides.

Other oils suitable for oxidation compositions (Ox) which are particularly preferred as contemplated herein are selected from the dicarboxylic acid esters of linear or branched C₂-C₁₀ alkanols, especially diisopropyl adipate, di-n-butyl adipate, di-(2-ethylhexyl) adipate, dioctyl adipate, diethyl/di-n-butyl/dioctyl sebacate, diisopropyl sebacate, dioctyl malate, dioctyl maleate, dicaprylyl maleate, diisooctyl succinate, di-2-ethylhexyl succinate and di-(2-hexyldecyl) succinate.

Other oils suitable for oxidation compositions (Ox) preferably used as contemplated herein are selected from the esters of linear or branched saturated fatty alcohols having 2-30 carbon atoms with linear or branched saturated fatty acids having 2-30 carbon atoms, which may be hydroxylated. These include preferably 2-hexyl decyl stearate, 2-hexyl decyl laurate, isodecyl neopentanoate, isononyl isononanoate, 2-ethylhexyl palmitate and 2-ethylhexyl stearate, isopropyl myristate, isopropyl palmitate, isopropyl stearate, isopropyl isostearate, isooctyl stearate, isonononyl stearate, isocetylstearate, isonononylisononanoate, isotridecylisononanoate, cetearylisononanoate, 2-ethylhexyl laurate, 2-ethylhexyl isostearate, 2-ethylhexyl cocoate, 2-octyldodecyl palmitate, butyloctanoic acid 2-butyloctanoate, diisotridecyl acetate, n-butyl stearate and n-hexyl laurate.

Other oils suitable for oxidation compositions (Ox), which as contemplated herein are particularly preferred, are selected from the C₈-C₂₂ fatty alcohol esters of monovalent or polyvalent C₂-C₇ hydroxycarboxylic acids, especially the esters of glycolic acid, lactic acid, malic acid, tartaric acid, citric acid and salicylic acid, e.g. C₁₂-C₁₅ alkyl lactate.

Other oils suitable for oxidation compositions (Ox) particularly preferred as contemplated herein are selected from the symmetrical, asymmetrical or cyclic esters of carbonic acid with C₃₋₂₂ alkanols, C₃₋₂₂ alkanediols or C₃₋₂₂ alkanetriols, e.g. dicaprylylcarbonate, or the esters according to DE19756454 A1, especially glycerine carbonate.

Preferred oxidizing compositions (Ox) used as contemplated herein contain at least one oil selected from the benzoic acid esters of linear or branched C₈₋₂₂ alkanols; triglycerides of linear or branched, saturated, optionally hydroxylated C₈₋₃₀ fatty acids; the dicarboxylic acid esters of linear or branched C₂-C₁₀ alkanols; the esters of linear or branched saturated fatty alcohols having 2-30 carbon atoms with linear or branched saturated fatty acids having 2-30 carbon atoms, which may be hydroxylated; the C₈-C₂₂ fatty alcohol esters of monovalent or polyvalent C₂-C₇ hydroxycarboxylic acids; the symmetrical, asymmetrical or cyclic esters of carbonic acid with C₃₋₂₂ alkanols, C₃₋₂₂ alkanediols or C₃₋₂₂ alkanetriols; the esters of dimers of unsaturated C₁₂-C₂₂ fatty acids (dimer fatty acids) with monovalent linear, branched or cyclic C₂-C₁₈ alkanols or with polyvalent linear or branched C₂-C₆ alkanols, and mixtures of the above substances.

Fat components with a melting point in the range of from about 23-110° C. preferably used in the oxidation compositions (Ox) as contemplated herein are selected from linear saturated 1-alkanols with 12-30 carbon atoms, preferably in a total amount of from about 0.1-8 wt. %, particularly preferably from about 3.0 to about 6.0 wt. %, each based on the weight of the oxidation composition (Ox) used as contemplated herein.

Preferably the at least one linear saturated 1-alkanol with 12-30 carbon atoms is selected from lauryl alcohol, myristyl alcohol, cetyl alcohol, stearyl alcohol, arachidyl alcohol, and behenyl alcohol as well as mixtures of these 1-alkanols, especially preferably cetyl alcohol, stearyl alcohol and cetyl alcohol/stearyl alcohol mixtures.

Oxidation compositions (Ox) preferably used as contemplated herein further contain, in each case based on their weight, at least one linear saturated 1-alkanol having 12-30 carbon atoms in a total amount of from about 0.1-8% by weight, preferably in a total amount of from about 2-6% by weight, where the at least one 1-alkanol may be selected from cetyl alcohol, stearyl alcohol and cetyl alcohol/stearyl alcohol mixtures.

Other oxidation compositions (Ox), which are preferably used as contemplated herein, contain at least one fat component with a melting point in the range of 23-110° C., which is selected from esters of a saturated, monovalent C₁₆-C₆₀ alkanol and a saturated C₈-C₃₆ monocarboxylic acid, in particular cetyl behenate, stearyl behenate and C₂₀-C₄₀ alkyl stearate, glycerol triesters of saturated linear C₁₂-C₃₀ carboxylic acids, which may be hydroxylated, candelilla wax, carnauba wax, beeswax, saturated linear C₁₄-C₃₆ carboxylic acids and mixtures of the above substances.

Other oxidation compositions (Ox) preferably used as contemplated herein contain at least one surfactant or at least one emulsifier, preferably in a total amount of from about 0.5-10% by weight, preferably from about 1-5% by weight, each based on the weight of the oxidation composition (Ox) used as contemplated herein.

For the purposes of the present application, surfactants and emulsifiers are amphiphilic (bifunctional) compounds which include at least one hydrophobic and at least one hydrophilic part of the molecule. The hydrophobic residue is preferably a hydrocarbon chain with 8-28 carbon atoms, which can be saturated or unsaturated, linear, or branched. This C₈-C₂₈-alkyl chain is particularly preferably linear. Basic properties of surfactants and emulsifiers are the oriented absorption at interfaces as well as the aggregation to micelles and the formation of lyotropic phases.

As contemplated herein, anionic, non-ionic, and cationic surfactants are particularly suitable. However, zwitterionic and amphoteric surfactants are also very suitable.

All anionic surfactants suitable for use on the human body are suitable as anionic surfactants in the compositions as contemplated herein. These are exemplified by a water-solubilizing anionic group such as a carboxylate, sulfate, sulfonate or phosphate group and a lipophilic alkyl group with 8 to 30 C atoms. In addition, glycol or polyglycol ether groups, ester, ether and amide and hydroxyl groups may also be present in the molecule. Examples of suitable anionic surfactants are linear and branched fatty acids with 8 to 30 C atoms (soaps), alkyl ether carboxylic acids, acyl sarcosides, acyl taurides, acyl isethionates, sulfosuccinic acid mono-, dialkyl esters and sulfosuccinic acid mono-alkyl polyoxyethyl esters, linear alkane sulfonates, linear alpha-olefin sulfonates, alkyl sulfates and alkyl ether sulfates and alkyl and/or alkenyl phosphates. Preferred anionic surfactants are alkyl sulfates, alkyl ether sulfates and alkyl ether carboxylic acids each with 10 to 18 C atoms, preferably 12 to 14 C atoms in the alkyl group and up to 12 glycol ether groups, preferably 2 to 6 glycol ether groups in the molecule. Examples of such surfactants are the compounds with the INCI designations sodium laureth sulfates, sodium lauryl sulfates, sodium myreth sulfates or sodium laureth carboxylates.

Zwitterionic surfactants are surface-active compounds which carry at least one quaternary ammonium group and at least one carboxylate, sulfonate or sulfate group in the molecule. Particularly suitable zwitterionic surfactants are the so-called betaines such as the N-alkyl-N,N-dimethylammonium-glycinate, for example the cocoalkyl-dimethylammoniumglycinate, N-acylaminopropyl-N,N-dimethylammoniumglycinate, for example, cocoacylaminopropyl dimethyl ammonium glycinate, and 2-alkyl-3-carboxymethyl-3-hydroxyethyl imidazolines each having 8 to 18 C atoms in the alkyl or acyl group, and cocoacylaminoethyl hydroxyethyl carboxymethyl glycinate. A preferred zwitterionic surfactant is the fatty acid amide derivative known under the INCI name cocamidopropyl betaine.

Amphoteric surfactants are surface-active compounds which, apart from a C₈-C₂₄ alkyl or acyl group, contain at least one free amino group and at least one —COOH or —SO₃H group in the molecule and can form internal salts. Examples of suitable amphoteric surfactants are N-alkylglycines, N-alkylpropionic acids, N-alkylaminobutyric acids, N-alkyliminodipropionic acids, N-hydroxyethyl-N-alkylamidopropylglycine, N-alkyltaurines, N-alkylsarcosines, 2-alkylaminopropionic acids and alkylaminoacetic acids each with 8 to 24 C atoms in the alkyl group. Particularly preferred amphoteric surfactants are N-cocoalkylaminopropionate, cocoacylaminoethylaminopropionate and C₁₂-C₁₈-Acylsarcosine.

Nonionic surfactants contain e.g. a polyol group, a polyalkylene glycol ether group or a combination of polyol and polyglycol ether group as the hydrophilic group. Such compounds are, for example, addition products of 4 to 50 mol ethylene oxide and/or 0 to 5 mol propylene oxide to linear and branched fatty alcohols, to fatty acids and to alkylphenols, each with 8 to 20 C atoms in the alkyl group, ethoxylated mono-, di- and triglycerides, such as glycerol monolaurate +20 ethylene oxide, and glycerol monostearate +20 ethylene oxide, sorbitan fatty acid esters and adducts of ethylene oxide with sorbitan fatty acid esters such as polysorbates (Tween 20, Tween 21, Tween 60, Tween 61, Tween 81), adducts of ethylene oxide with fatty acid alkanolamides and fatty amines, and alkyl polyglycosides. C₈-C₂₂ alkyl mono- and oligoglycosides and their ethoxylated analogues and ethylene oxide addition products to saturated or unsaturated linear fatty alcohols with 2 to 30 moles of ethylene oxide per mole of fatty alcohol are particularly suitable as nonionic surfactants.

Further oxidation compositions preferably used as contemplated herein at least one anionic surfactant selected from alkyl sulfates, alkyl ether sulfates and alkyl ether carboxylic acids each having 10 to 18 C-atoms, preferably 12 to 14 C atoms in the alkyl group and up to 12, preferably 2 to 6, glycol ether groups in the molecule.

Other oxidation compositions preferably used as contemplated herein include at least one nonionic surfactant selected from ethylene oxide adducts to saturated or unsaturated linear fatty alcohols with 2 to 30 moles of ethylene oxide per mole of fatty alcohol, and at least one anionic surfactant selected from alkyl sulfates, alkyl ether sulfates and alkyl ether carboxylic acids each having 10 to 18 C atoms, preferably 12 to 14 C atoms in the alkyl group and up to 12, preferably 2 to 6 glycol ether groups, in the molecule, the weight ratio of the total of all anionic surfactants to the total of all nonionic surfactants being particularly preferably in the range of from about 5-50, preferably from about 10-30.

In principle, all cationic surface-active substances suitable for use on the human body are suitable as cationic surfactants in oxidation compositions (Ox) which are preferably used as contemplated herein. These are exemplified by at least one water-solubilizing, cationic group, such as a quaternary ammonium group, or by at least one water-solubilizing, cationizable group, such as an amine group, and furthermore at least one (lipophilically acting) alkyl group with 6 to 30 C atoms or at least one (lipophilically acting) imidazole group or at least one (lipophilically acting) imidazylalkyl group.

Oxidation compositions (Ox) which are used with particular preference as contemplated herein contain at least one cationic surfactant which is preferably selected from quaternary ammonium compounds having at least one C8-C24 alkyl radical, esterquats and amidoamines each having at least one C8-C24 acyl radical, and mixtures thereof. Preferred quaternary ammonium compounds with at least one C8-C24 alkyl radical are ammonium halides, especially chlorides, and ammonium alkyl sulfates, such as methosulfates or ethosulfates, such as C8-C24 alkyl trimethylammonium chlorides, C8-C24 dialkyldimethylammonium chlorides and C8-C24 trialkylmethylammonium chlorides, e.g. cetyltrimethylammonium chloride, stearyltrimethylammonium chloride, distearyldimethylammonium chloride, lauryldimethylammonium chloride, lauryldimethylbenzylammonium chloride and tricetylmethylammonium chloride, as well as the imidazolium compounds known under the INCI designations Quaternium-27, Quaternium-83, Quaternium-87 and Quaternium-91. The alkyl chains of the surfactants mentioned above preferably have 8 to 24 carbon atoms.

Esterquats are cationic surfactants which contain both at least one ester function and at least one quaternary ammonium group as a structural element and at least one C8-C24 alkyl or C8-C24 acyl radical. Preferred esterquats are quaternized ester salts of fatty acids with triethanolamine, quaternized ester salts of fatty acids with diethanolalkylamines and quaternized ester salts of fatty acids with 1,2-dihydroxypropyl dialkylamines N,N-bis(2-palmitoyloxyethyl)dimethylammonium chloride, distearoylethyl dimonium methosulfates and distearoylethyl hydroxyethylmonium methosulfates are preferred examples of such esterquats.

Alkylamidoamines are usually produced by amidation of natural or synthetic C8-C24 fatty acids and fatty acid sections with di-(C1-C3)alkylaminoamines A compound from this substance group which is particularly suitable as contemplated herein is stearamidopropyldimethylamine.

Oxidation compositions (Ox) used with particular preference as contemplated herein contain at least one cationic surfactant in a total amount of from about 0.01-5% by weight, preferably from about 0.1-3% by weight, particularly preferably from about 0.3-2% by weight, each based on the weight of the oxidation composition (Ox) used as contemplated herein.

A further object of the present disclosure is a multi-component packaging unit (kit-of-parts) for brightening keratinous fibers, which contains at least two separately packaged components, and in which:

i) the first component (I) is a blonding powder as contemplated herein or a blonding powder preferred as contemplated herein;

ii) the second component (II) is an oxidation composition containing, in each case based on its weight, from about 50-96%, preferably from about 70-93%, particularly preferably from about 80-90%, by weight of water and from about 0.5-20% by weight of hydrogen peroxide and having a pH in the range of from about 1.5 to about 5.5, measured at about 20° C., where components (I) and (II) are preferably in a weight-related ratio (I) : (II) of from about 0.2-1, particularly preferred from about 0.3-0.8, further preferred from about 0.4-0.7, extremely preferred from about 0.5-0.6.

The ready-to-use mixtures of a blonding powder according to the present disclosure with one of the aforementioned oxidation compositions (Ox) preferably have a viscosity in the range of from about 15,000 to about 100,000 mPas, particularly preferably from about 20,000 to about 85,000 mPas, each measured at about 20° C. with a Brookfield viscometer type DV-II+, spindle 5 at a speed of about 4 rpm. A viscosity in this range allows the ready-to-use mixture to be easily applied on the one hand and on the other hand to have such a flow behavior that it guarantees a sufficiently long exposure time for the agent to act on the keratinic fibers at the place of action.

A multi-component packaging unit comprises several individual components that are assembled separately from each other, as well as a common packaging for these components, for example a folding box. Here the components are provided separately in different containers. In the context of the present disclosure, a container is understood to be an enclosure in the form of a bottle, a tube, a can, a sachet, a sachet, or a similar wrapping, which may be re-closable. According to the present disclosure there are no limits to the wrapping material. Preferably, however, these are wrappings made of glass or plastic.

In addition, the packaging unit may include application aids such as combs, brushes or paint brushes, personal protective clothing, especially disposable gloves, and instructions for use.

Furthermore, the blonding powders as contemplated herein or preferred in the present disclosure may contain at least one direct dye. These are dyes that are applied directly to the hair and do not require an oxidative process to form the color. For matting undesirable residual color impressions caused by melanin decomposition products, especially in the reddish or bluish area, certain direct dyes of complementary colors are particularly preferred. Direct dyes are usually nitrophenylenediamines, nitroaminophenols, azo dyes, anthraquinones or indophenols. Direct dyes can be anionic, cationic, or non-ionic. The direct-drawing dyes are each preferably contained in an amount of from about 0.001 to about 2% by weight, based on the weight of the bleaching powder.

Preferred anionic direct dyes are the compounds known under the international names or trade names Acid Yellow 1, Yellow 10, Acid Yellow 23, Acid Yellow 36, Acid Orange 7, Acid Red 33, Acid Red 52, Pigment Red 57:1, Acid Blue 7, Acid Green 50, Acid Violet 43, Acid Black 1, Acid Black 52, Bromphenol Blue and Tetrabromophenol Blue. Preferred cationic direct dyes are cationic triphenylmethane dyes, for example Basic Blue 7, Basic Blue 26, Basic Violet 2 and Basic Violet 14, aromatic systems substituted with a quaternary nitrogen group, such as Basic Yellow 57, Basic Red 76, Basic Blue 99, Basic Brown 16 and Basic Brown 17, cationic anthraquinone dyes such as HC Blue 16 (Bluequat B) and direct dyes containing a heterocycle containing at least one quaternary nitrogen atom, in particular Basic Yellow 87, Basic Orange 31 and Basic Red 51. The cationic direct dyes marketed under the trademark Arianor® are, as contemplated herein, also preferred cationic direct dyes. Non-ionic direct dyes are particularly suitable as non-ionic direct dyes, such as nitro and quinone dyes and neutral azo dyes. Preferred non-ionic direct dyes are those under the international designations or Trade names HC Yellow 2, HC Yellow 4, HC Yellow 5, HC Yellow 6, HC Yellow 12, HC Orange 1, Disperse Orange 3, HC Red 1, HC Red 3, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9 known compounds, as well as 1,4-diamino-2-nitrobenzene, 2-amino-4-nitrophenol, 1,4-bis-(2-hydroxyethyl)-amino-2-nitrobenzene, 3-nitro-4-(2-hydroxyethyl)aminophenol 2-(2-hydroxyethyl)amino-4,6-dinitrophenol, 4-[(2-hydroxyethyl)amino]-3-nitro-1-methylbenzene, 1-amino-4-(2-hydroxyethyl)amino-5-chloro-2-nitrobenzene, 4-amino-3-nitrophenol, 1-(2′-ureidoethyl)amino-4-nitrobenzene, 2-[(4-amino-2-nitrophenyl)amino]benzoic acid, 6-nitro-1,2,3,4-tetrahydroquinoxaline, 2-hydroxy-1,4-naphthoquinone, picramic acid and its salts, 2-amino-6-chloro-4-nitrophenol, 4-ethylamino-3-nitrobenzoic acid and 2-chloro-6-ethylamino-4-nitrophenol. A combination of tetrabromophenol blue and Acid Red 92 is particularly preferred.

Preferably the exposure time is from about 5 to about 60 min, especially from about 5 to about 50 min, especially preferably from about 10 to about 45 min. During the exposure time of the agents on the fiber it can be advantageous to support the whitening process by adding heat. An exposure phase at room temperature is also according to present disclosure. In particular, the temperature during the exposure time is between about 20° C. and about 40° C., especially between about 25° C. and about 38° C. The agents give good treatment results even at physiologically compatible temperatures below about 45° C.

At the end of the whitening process, all components found on the keratin fibers are rinsed from the hair with water or a surfactant-based detergent. In particular, commercially available shampoo can be used as a cleaning agent, whereby the cleaning agent can be dispensed with and the rinsing process can be carried out with tap water if the whitening agent has a higher surfactant content.

What has been said about the blonding powders as contemplated herein and preferred in the present disclosure applies mutatis mutandis also to the multi-component packaging units (kits of parts) as contemplated herein and preferred in the present disclosure.

What has been said about the blonding powders as contemplated herein and preferred in the present disclosure applies mutatis mutandis also to the processes for brightening keratinous fibers as contemplated herein and preferred in the present disclosure.

What has been said about the oxidation compositions used as contemplated herein and preferred in the present disclosure also applies mutatis mutandis to the multi-component packaging units (kits of parts) used as contemplated herein and preferred in the present disclosure.

What has been said about the oxidation compositions used as contemplated herein and preferred in the present disclosure also applies mutatis mutandis to the process for brightening keratinous fibers as contemplated herein and preferred in the present disclosure.

What has been said about the blonding powders as contemplated herein and preferred as contemplated herein applies mutatis mutandis also to the use as contemplated herein.

What is said about the oxidation compositions used preferentially as contemplated herein also applies mutatis mutandis to the use as contemplated herein.

### EXAMPLES

1.1 Developer Emulsion (Ox)

| Ingredient | Quantity (% by weight) |
|---|---|
| Dipicolinic acid (2,6- | 0.1 |
| dicarboxypyridine) | |
| Potassium hydroxide | 0.13 |
| Etidronic acid | 0.24 |
| Sodium cetaryl sulfate | 0.3 |
| Cetearyl alcohol | 3.6 |
| Ceteareth-20 | 0.5 |
| PEG-40 Castor Oil | 0.6 |
| Isopropyl myristate | 10.0 |
| Disodium pyrophosphate | 0.1 |
| Cera Alba (Beeswax, Cire d'abeille) | 0.3 |
| Hydrogen peroxide | 5.54 |
| Water | ad 100 |

1.2 Blonding powder formulations according to the present disclosure (unless otherwise stated, the quantities correspond to weight-%)

| | No. 1.2.1 | No. 1.2.2 | No. 1.2.3 | No. 1.2.4 |
|---|---|---|---|---|
| Sodium metasilicate | 4.1 | 4.5 | 4.7 | 6.0 |
| Sodium silicate | 17.6 | 17.6 | 17.6 | 17.6 |
| Magnesium carbonate | 34.5 | 41.8 | 27.85 | 13.75 |
| hydroxide | | | | |
| Cellulose Gum | 2.8 | 2.5 | 2.5 | 2.5 |
| Hydroxyethyl cellulose | 0.7 | 0.35 | 0.65 | 0.85 |
| Tetrasodium EDTA | 0.4 | 0.4 | 0.5 | 0.6 |
| Potassium persulfate | 14.4 | 15 | 18 | 21 |
| Silica | 3.0 | 3 | 3 | 3 |
| Potassium sulfate | 0.2 | 0.2 | 0.3 | 0.4 |
| Sodium sulfate | 0.1 | 0.05 | 0.1 | 0.2 |
| Sodium persulfate | 8.5 | 4 | 10 | 15 |
| Polyquaternium-4 | 0.1 | 0.2 | 0.3 | 0.4 |
| Octyldodecanol | 11.6 | 8 | 11 | 14.1 |
| Dicaprylyl Ether | 2.0 | 2.4 | 3.5 | 4.6 |
| | 100.0 | 100.0 | 100.0 | 100.0 |

The blonding powder and developer emulsion were mixed in a weight ratio of 1:2.

2. Application

100 g of the freshly prepared mixture of the bleaching powder and developer emulsion were applied to dry hair strands (dark blond (Kerling 6/0)) (4 g application mixture per gram of hair). After the strands were bleached for 45 minutes at 35° C., they were washed with water for 2 minutes and dried with a hairdryer.

3. Whitening Performance

By colorimetric measurement with a colorimeter, the delta L value (L_{Bleached strands}-L_{bleached area}) as a measure of the whitening performance

| | Blonding powder 1.2.1 with | Blonding powder 1.2.2 with |
|---|---|---|
| | developer | developer |
| ΔL | 31.2 | 32.5 |

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A blonding powder comprising:
a) at least one oxidizing agent selected from the group of sodium salts of a peroxosulfuric acid, potassium salts of a peroxosulfuric acid, and mixtures thereof,
b) from about 0.1 to about 15% by weight of at least one branched fatty alcohol with 8-24 carbon atoms, based on a weight of the blonding powder,
c) from about 0.1 to about 15% by weight of at least one dialkyl ether with 6 to 18 C atoms in the alkyl group, based on the weight of the blonding powder,
d) at least one inorganic alkalizing agent which is solid at about 20° C. and 1013 mbar, and
e) from 0 to about 8% by weight of water, based on the weight of the blonding powder,
f) wherein a total amount of oils, including components b) and c), is from about 0.2 to about 20% by weight, based on the weight of the blonding powder, and
wherein the blonding powder comprises no ammonium compounds, no paraffin oil, no mineral oil, and no silicone compounds.

2. The blonding powder according to claim 1, wherein the at least one oxidizing agent a) is included in a total amount of from about 5 to about 85% by weight, based on the weight of the blonding powder.

3. The blonding powder according to claim 1, wherein the at least one inorganic alkalizing agent which is solid at about 20° C. and 1013 mbar comprises at least one sodium silicate or sodium metasilicate with a molar SiO₂/Na₂O ratio of ≥2, wherein the at least one inorganic alkalizing agent is included in a total amount of from about 0.1 to about 50% by weight, based on the weight of the blonding powder.

4. The blonding powder according to claim 1, wherein the at least one dialkyl ether having 6 to 18 C atoms in the alkyl group is selected from the group of di-n-alkyl ethers having a total of 12 to 36 C atoms.

5. The blonding powder according to claim 4, wherein the at least one dialkyl ether having 6 to 18 C atoms in the alkyl group is selected from the group of di-n-octyl ether, di-n-decyl ether, di-n-nonyl ether, di-n-undecyl ether, di-n-dodecyl ether, di-n-octadecyl ether, n-hexyl-n-octyl ether, n-octyl-n-decyl ether, n-decyl-n-undecyl ether, n-undecyl-n-dodecyl ether, n-hexyl-n-undecyl ether, di-(2-ethylhexyl) ether, di-(2-ethyldecyl) ether, and mixtures thereof.

6. The blonding powder according to claim 1, wherein the at least one branched fatty alcohol with 8-24 carbon atoms is selected from the group of 2-octyldodecanol, 2-hexyldecanol, 2-ethylhexyl alcohol, isostearyl alcohol, and mixtures thereof.

7. The blonding powder according to claim 1, wherein the at least one oxidizing agent a) is selected from the group of sodium peroxodisulfate, potassium peroxodisulfate, sodium percarbonate, sodium peroxomonosulfate, potassium peroxomonosulfate, and mixtures of these compounds.

8. The blonding powder according to claim 1, wherein the total amount of oils, including components b) and c), is from about 1 to about 17% by weight, based on the weight of the blonding powder.

9. A method for lightening keratinous fibers, the method comprising the steps of:
mixing a blonding powder (B) with an oxidizing composition (Ox) to form a ready-to-use mixture, wherein the blonding powder (B) comprises a) at least one oxidizing agent selected from the group of sodium salts of a peroxosulfuric acid, potassium salts of a peroxosulfuric acid, and mixtures thereof, b) from about 0.1 to about 15% by weight of at least one branched fatty alcohol with 8-24 carbon atoms, based on a weight of the blonding powder (B), c) from about 0.1 to about 15% by weight of at least one dialkyl ether with 6 to 18 C atoms in the alkyl group, based on the weight of the blonding powder (B), d) at least one inorganic alkalizing agent which is solid at about 20° C. and 1013 mbar, and e) from 0 to about 8% by weight of water, based on the weight of the blonding powder (B), wherein a total amount of oils, including components b) and c), is from about 0.2 to about 20% by weight, based on the weight of the blonding powder (B), and wherein the blonding powder (B) comprises no ammonium compounds, no paraffin oil, no mineral oil, and no silicone compounds, wherein the oxidizing composition (Ox) comprises, based on its weight, from about 50-96% by weight water, 0.5-20% by weight of hydrogen peroxide, and at least one pH adjusting agent in such an amount that the oxidation composition (Ox) has a pH value in the range from about 1.5 to about 5.5, measured at about 20° C.,
applying the ready-to-use mixture to the keratinous fibers,
leaving the ready-to-use mixture on the keratinous fibers for from about 5 to about 60 minutes, and
rinsing the keratinous fibers with water and optionally with a surfactant-containing cleaning agent, and
wherein the blonding powder (B) and the oxidizing composition (Ox) are in a weight ratio (B) : (Ox) of from about 0.2 to about 1.

10. The method of claim 9, wherein the weight ratio (B) : (Ox) is from about 0.3 to about 0.8.

11. The method of claim 9, wherein the weight ratio (B) : (Ox) is from about 0.3 to about 0.7.

12. The method of claim 9, wherein the at least one oxidizing agent a) is included in the blonding powder (B) in a total amount of from about 5 to about 85% by weight, based on the weight of the blonding powder.

13. The method of claim 9, wherein the at least one inorganic alkalizing agent which is solid at about 20° C. and 1013 mbar comprises at least one sodium silicate or sodium metasilicate with a molar SiO₂/Na₂O ratio of ≥2, wherein the at least one inorganic alkalizing agent is included in the blonding powder (B) in a total amount of from about 0.1 to about 50% by weight, based on the weight of the blonding powder (B).

14. The method of claim 9, wherein the at least one dialkyl ether having 6 to 18 C atoms in the alkyl group is selected from the group of di-n-alkyl ethers having a total of 12 to 36 C atoms.

15. The method of claim 9, wherein the at least one dialkyl ether having 6 to 18 C atoms in the alkyl group is selected from the group of di-n-octyl ether, di-n-decyl ether, di-n-nonyl ether, di-n-undecyl ether, di-n-dodecyl ether, di-n-octadecyl ether, n-hexyl-n-octyl ether, n-octyl-n-decyl ether, n-decyl-n-undecyl ether, n-undecyl-n-dodecyl ether, n-hexyl-n-undecyl ether, di-(2-ethylhexyl) ether, di-(2-ethyldecyl) ether, and mixtures thereof.

16. The method of claim 9, wherein the at least one branched fatty alcohol with 8-24 carbon atoms is selected from the group of 2-octyldodecanol, 2-hexyldecanol, 2-ethylhexyl alcohol, isostearyl alcohol, and mixtures thereof.

17. The method of claim 9, wherein the at least one oxidizing agent a) is selected from the group of sodium peroxodisulfate, potassium peroxodisulfate, sodium percarbonate, sodium peroxomonosulfate, potassium peroxomonosulfate, and mixtures of these compounds.

18. The method of claim 9, wherein the total amount of oils in the blonding powder (B), including components b) and c), is from about 1 to about 17% by weight, based on the weight of the blonding powder (B).

19. The blonding powder of claim 1, wherein the blonding powder comprises water in an amount of from about 0.5 to about 3% by weight, based on the weight of the blonding powder.

20. The blonding powder of claim 1, wherein the at least one oxidizing agent a) is included in the blonding powder in a total amount of from about 22 to about 45% by weight, based on the weight of the blonding powder.
